(19) United States
(12) Patent Application Publication
(10) Pub. No.: US 2003/0207856 A1
(43) Pub. Date: Nov. 6, 2003

(54) Medical devices and compositions for delivering anti-proliferatives to anatomical sites at risk for restenosis

(76) Inventors: Patrice Tremble, Santa Rosa, CA (US); Marc Hendriks, Brunssum (NL); Wenda Carlyle, Silverado, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/392,229

(22) Filed: Mar. 18, 2003

Related U.S. Application Data
(60) Provisional application No. 60/365,497, filed on Mar. 18, 2002.

(51) Int. Cl. A61M 29/00; A61K 31/33

(52) U.S. Cl. 514/183; 604/96.01

(57) ABSTRACT

Methods, compositions and devices for inhibiting restenosis are provided. Specifically, molecular chaperone inhibitor compositions and medical devices useful for the site specific delivery of molecular chaperones are disclosed. In one embodiment the medical device is a vascular stent coated with a molecular chaperone inhibitor selected from the group consisting of geldanamycin, herbimycin, macbecin and derivatives and analogues thereof. In another embodiment an injection catheter for delivery an anti-restenotic effective amount of geldanamycin to the adventitia is provided.

### RELATED APPLICATIONS

This application claims priority to U.S. Provisional Patent Application No. 60/365,497, filed Mar. 18, 2002.

### FIELD OF THE INVENTION

The present invention relates to medical devices and compositions for treating or preventing restenosis. Specifically, the present invention relates the site specific delivery of anti-proliferative compounds using a medical device. More specifically, the present invention relates to devices for delivering molecular chaperone inhibitors to regions of the mammalian vasculature at risk for restenosis.

### BACKGROUND OF THE INVENTION

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is, a percutaneous procedure wherein a balloon catheter is inserted into the coronary artery and advanced until the vascular stenosis is reached. The balloon is then inflated restoring arterial patency. One angioplasty variation includes arterial stent deployment. Briefly, after arterial patency has been restored, the balloon is deflated and a vascular stent is inserted into the vessel lumen at the stenosis site. The catheter is then removed from the coronary artery and the deployed stent remains implanted to prevent the newly opened artery from constricting spontaneously. However, balloon catheterization and stent deployment can result in vascular injury ultimately leading to VSMC proliferation and neointimal formation within the previously opened artery. This biological process whereby a previously opened artery becomes re-occluded is referred to as restenosis.

Treating restenosis requires additional, generally more invasive, procedures including CABG in some cases. Consequently, methods for preventing restenosis, or treating incipient forms, are being aggressively pursued. One possible method for preventing restenosis is the administration of medicaments that block local invasion/activation of monocytes thus preventing the secretion of growth factors that may trigger VSMC proliferation and migration. Metabolic inhibitors such as anti-neoplastic agents are currently being investigated as potential anti-restenotic compounds. However, the toxicity associated with the systemic administration of metabolic inhibitors has recently stimulated research into in situ, site-specific drug delivery.

Anti-restenotic coated stents are one potential method of site-specific drug delivery. Once the coated stent is deployed, it releases the anti-restenotic agent directly into the tissue thus allowing for clinically effective drug concentrations to be achieved locally without subjecting the recipient to side effects associated with systemic drug delivery. Moreover, localized delivery of anti-proliferative drugs directly at the treatment site eliminates the need for specific cell targeting technologies.

Recently, significant research has been conducted utilizing compounds that inhibit cell cycle progression or completion. For convenience the mammalian cell cycle has been divided into four discrete segments. Mitosis and cell division occur in the M phase which lasts for only about one hour. This is followed by the G₁ phase (G for Gap) and then the S phase (S for syntheses) during which time DNA is replicated, and finally G₂ phase during which the cell prepares for mitosis. Eukaryotic cells in culture typically have cell cycle times of 16-24 hours; however, in some multicellular organisms the cell cycle can last for over 100 days. Furthermore, some cells such as neurons stop dividing completely in the mature mammal and are considered to be quiescent. This phase of the cell cycle is often referred to as G₀.

Variations in non-quiescence cell cycle times are largely dependent on the duration of the G₁ phase. Therefore, it is logical that a significant number of antiproliferative cell cycle inhibitors target cellular functions occurring during G₁. However, cell cycle inhibition is not limited to agents that selectively target the G₁ phase. For example, a number of cytotoxic compounds that either inhibit mitotic spindle formation or mitotic spindle separation are known. These compounds, such as paclitaxol target the M phase of the cell cycle. Compounds that affect DNA syntheses such as DNA topisomerases inhibitors block cell proliferation during the G₂ and S phase. However, regardless of the cell cycle phase affected, antiproliferative compounds target dividing cells and leave quiescent cells essentially undisturbed. This theory underlies the development of most anti-cancer chemotherapeutics.

Regardless of which phase a proliferating cell is in, protein turnover is an essential process. The post transnational processing of proteins including folding, intracellular transport and degradation are mediated by a family of heat shock (Hsp) proteins known as molecular chaperones (Smith D. F. et al. 1998. Molecular Chaparones: Biology and Prospects for Pharmacological Intervention. Pharm. Rev. Vol. 50, No 4; 493-513). Proteins assume their post transnational configuration through intra-peptide chain interactions between hydrophobic and hydrophilic regions. As newly synthesized peptides emerge from the ribosome the hydrophobic and hydrophilic regions are exposed to the intracellular environment including other recently translated regions of the same polypeptide chain Martain J. and F. U. Hartl. 1997. Chaperone-assisited protein folding. Curr. Opin. Struct. Biol.7:41-52).

In the absence of a molecular intermediate such as a chaperone, portions of the nascent polypeptide chain could interact non-specifically resulting in denatured, non-functional proteins (Id). Furthermore, molecular chaperones are also essential for transporting recently synthesized native proteins throughout the intracellular milieu. For example, chaperones directly participate in transporting proteins across cell membranes including the mitochondrial membrane. Mitochondrial proteins encoded for by nuclear genes must be transported from intracellular ribosomes through the cytoplasm and across the mitochondrial membrane where they are refolded through the combined actions of cellular and mitochondrial chaperones (Langer, T, et al. 1997. Functions of molecular chaperone proteins in biogenesis of mitochondria, In: Guidebook to Molecular Chaperones and Protein Folding Catalysts (Gething M -J ed) pp 499-506, Oxford University Press, Oxford, U.K.).

Molecular chaperones are divided into families based on their approximate molecular weights. In eukaryotic systems the major Hsps are Hsp 100, Hsp 90, Hsp 70, Hsp 60, Hsp 40, Hsp and a family of smaller Hsps ranging in molecular weight between 20 to 25 kilodaltons. Although the exact function of each family is still being elucidated, it is understood that nascent peptide chain folding is principally mediated by Hsp 70, Hsp 60 and Hsp 40. The smaller Hsps interact with misfolded proteins and facilitate their desegregation and degradation. Many Hsps have functions beyond participating in protein folding and degradation. For example, Hsp 70 plays a critical role in protein membrane translocation, and recently Hsp 90 has been identified as an important regulatory protein. Members of the Hsp 100 family help prevent, and in some cases reverse, heat shock related protein aggregation and facilitate cells acquire thermotollerance (Hartl, F. U. 1996. Molecular Chaperones in cellular protein folding. Nature (Lond.) 38:571-580).

The most abundantly expressed Hsp in eukaryotic systems is Hsp 90. It's been established using Hsp 90 knock-out models that Hsp 90 expression is essential for cell survival and proliferation; however, Hsp 90 appears to have a minimal role in mediating nascent peptide chain folding. Therefore, significant research has been directed at understanding Hsp 90's contribution to normal cell metabolism. Recently, it has been established that Hsp 90 participates in several essential intracellular functions. Hsp 90 interacts with a wide range of regulatory proteins including transcription factors, tyrosine and serine/threonine kinases and steroid hormone receptors in addition to participating in denatured protein re-folding following heat shock (Eggers, D. K. et al. 1997 Complexes between nascent polypeptide and their molecular chaperones in the cytosol of mammalian cells. Mol. Bio. Cell. 8:1559-1573).

The molecular chaperone mediated activation and assembly of proteins involved in signal transduction, cell cycle control and transcriptional regulation is a complex pathway. Hsp 90 is central to this pathway which involves numerous Hsp 90 binding proteins including Hsp 70-binding proteins, the FK-506-binding proteins (FKBP) FKBP51, FKBP52, FKBP56, and FKBP59 (collectively referred to hereinafter as Hsp90-associated immunophilins). These Hsp 90-associated immunophilins possess peptidylpropyl isomerase (PPlase) activity. PPlases convert propyl residues within a polypeptide chain from a trans to a cis configuration which in turn accelerates protein folding and hence protein activation. Moreover, recently ATPase activity has been shown to be essential to Hsp90's in vivo activity (Nair S. C. et al. 1997. Molecular cloning of human FKBP51 and comparisons of immunophilin interactions with Hsp90 and progesterone receptor. Mol. Cell Biol. 17:594-603).

The recognition that cells having Hsp 90 knock-outs genes fail to proliferate has made Hsp 90 an attractive intracellular target for anti-proliferative chemotherapeutics. Furthermore, because many molecular chaperones including Hsp 90 are constitutively expressed, chaperone inhibitors can effectively inhibit cell proliferation at any point in the cell cycle. Therefore, molecular chaperones inhibitors are ideal antiproliferative candidates and may prove beneficial in treating or inhibiting restenosis. Consequently, it is an object of the present invention to provide medical devices and methods for the site specific delivery of molecular chaperone inhibitors to mammalian anatomical lumens at risk for restenosis.

### SUMMARY OF THE INVENTION

The present invention relates to medical devices and methods for treating or inhibiting restenosis. Specifically, the present invention relates to.devices for delivering molecular chaperone inhibitors to regions of the mammalian vasculature at risk for restenosis.

In one embodiment of the present invention a stent is adapted to deliver a molecular chaperone inhibitor directly to the tissue of a mammalian lumen at risk for developing restenosis.

In another embodiment of the present invention restenosis is treated or inhibited by administering an inhibitor of mammalian heat shock proteins (Hsp) directly to the tissue of a mammalian lumen at risk for developing restenosis.

In yet another embodiment of the present invention the molecular weight of the Hsp is selected from the group consisting of 100 kDa, 90 kDa, 70 kDa, 60 kDa, 40 kDa, 25 kDa, and 20 kDa.

In yet another embodiment of the present invention the Hsp is Hsp 90.

In still another embodiment of the present invention the molecular chaperone inhibitor is a benzoquinone ansamycin including geldanamycin.

In another embodiment of the present invention the stent adapted to deliver the molecular chaperone inhibitor is a vascular stent and the mammalian anatomical lumen is a blood vessel.

In yet another embodiment of the present invention the vascular stent is delivered to the site at risk for restenosis within a blood vessel using a balloon catheter.

In another embodiment of the present innovation an injection catheter is used to deliver chaperone inhibitors to the adventitia at or near a site of restenosis, or an area susceptible to restenosis.

### BRIEF DESCRIPTION OF THE FIGURES

FIG. 1 depicts a vascular stent used to deliver the antirestenotic compounds of the present invention.

FIG. 2 depicts a balloon catheter assembly used for angioplasty and the site-specific delivery of stents to anatomical lumens at risk for restenosis.

FIG. 3 depicts the needle of an injection catheter in the retracted position (balloon deflated) according to the principles of the present invention where the shaft is mounted on an intravascular catheter.

FIGS. 4 and 5 illustrate use of the apparatus of FIG. 3 in delivering a substance into the adventitial tissue surrounding a blood vessel.

FIG. 6 graphically depicts the effects of geldanamycin on HCASMC proliferation at four days.

FIG. 7 graphically depicts the percent inhibition of HCASMC proliferation as a function of geldanamycin concentration in nM.

FIG. 8 graphically depicts the in vitro fast elution profile of geldanamycin coated vascular stent.

FIG. 9 graphically depicts the intro slow elution profile of geldanamycin coated vascular stent.

FIG. 10 graphically compares various in vitro elution profiles of geldanamycin coated stents with in vivo elution profiles of geldanamycin coated stents.

FIG. 11 graphically depicts the correlation between neointimal thickness and injury score in the combined proximal and distal stent segments in test pigs.

### DETAILED DESCRIPTION OF THE INVENTION

As previously discussed, molecular chaperones are constitutively expressed regulatory proteins essential for normal cell metabolism and proliferation. Molecular chaperones help regulated protein turnover, post translational folding, protein transport, and function as regulatory factors for a variety of signaling proteins. Therefore, molecular chaperones are attractive targets for anti-proliferative chemotherapeutics.

Heat shock protein 90 (Hsp 90) is one of the most promising molecular chaparones being targeted by anti-proliferative compounds. (Neckers, L. et al. 1999. Geldanamycin as a potential anti-cancer agent: Is molecular target and biochemical activity. Invest. New Drugs. 17:361-373 and Yorgin P. D. et al. 2000. Effects of Geldanamycin, a heat shock protein 90-binding agent, on T cell function and T cell nonreceptor protein tyrosine kinases. J. Immunol. 164(6) 2915-2923). Hsp 90 associates with a variety of regulatory proteins including transcription factors, tyrosine and serine/threonine kinases and steroid hormone receptors, Hsp 70-binding proteins, FKBP51, FKBP52, FKBP56, and FKBP59 (the Hsp 90-associated immunophilins) (Cardenas, M. E. et al. 1998 Signal-transduction cascades as targets for therapeutic intervention by natural products. Trends Biotech. Oct., 16(10) 427-433). Hsp 90-associated immunophilins possess peptidylpropyl isomerase (PPlase) activity (Barent, R. L. et al. 1998.Analysis of FKBP51/FKBP52 chimeras and mutants for Hsp 90 binding and association with progesterone complexes. Mol. Endocrinol. 12:342-354). PPlases convert propyl residues within a polypeptide chain from a trans to a cis configuration which in turn accelerates protein folding and hence protein activation. Moreover, recently Hsp90 has been shown to possess ATPase activity and that this is essential to Hsp90's in vivo activity. These properties combine to make Hsp 90 a most attractive anti-proliferative target.

There are numerous compounds that can bind to and inhibit molecular chaparones including ansamycins and radicicol. The ansamycins all inhibit Hsp 90 by binding to an N-terminal ATP binding pocket. (Roe, S. M. et al. 1999 Structural basis for inhibition of the Hsp90 molecular chaperone by the antitumor antibiotics radicicol and geldanamycin. J. Med. Chem. 42:260-266). Radicicol, a non-ansamycin Hsp 90 inhibitor, was found to have the same mechanism of action (Id). Therefore, in searching for new inhibitors, compounds were selected based on the prediction of binding to this same site; one such compound is novobiocin. Surprisingly, despite its apparent effectiveness, it was found to act by binding to a site distal to the ATP binding domain. This serendipitous discovery suggests that screening based on Hsp 90 inhibition rather than a specific mechanism of inhibition is likely to be more fruitful.

However, any molecular chaperone inhibitor that inhibits or interferes with the normal biological function of any heat shock protein is within the scope of the present invention. As used herein, heat shock proteins include, but are limited to those having molecular weights including approximately 100 kDa, 90 kDa, 70 kDa, 60 kDa, 40 kDa, 25 kDa, 20 kDa and others.

In one embodiment of the present invention the ansamycin is a benzoquinone ansamycin and derivatives thereof. Many benzoquinone ansamycins occur naturally as fermentation products of Sterptomyces hygroscopicus. The best known naturally occurring benzoquinone ansamycin is the antiprotozoan antibiotic geldanamycin which was first characterized by DeBoer in 1970 (DeBoer C., et al. 1970. J. Antibiotics Vol. 23, page 442). For example, see U.S. Pat. Nos. 3,595,955 issued Jul. 27, 1971 to DeBoer et al., 4,261,989 issued Apr. 14, 1981 to Saski et al, 5,932,566 issued Aug. 3, 1999 to Schnur and 6,174,875 B1 issued Jan. 16, 2001 to DeFranco et al. In addition to geldanamycin there are several other naturally occurring benzoquinone ansamycins such as herbimycin and macbecin and a number of synthetically derives analogues and derivatives. The benzoquinoid ansamycins possess a benzoquinone moiety, an ansa ring, and a carbamate moiety and may be represented by the following general formula:

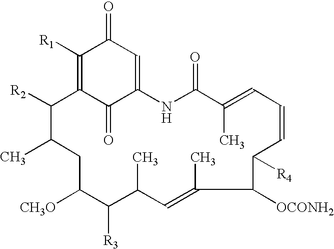

Where R₁═OCH₃; R₂═H, R₃═OH; R₄═OCH₃ the benzoquinoid ansamycin is geldanamycin; where R₁═H; R₂═OCH₃, R₃═OCH₃; R₄═OCH₃ the benzoquinoid ansamycin is herbimycin and where R₁═H; R₂═OCH₃, R₃═OCH₃; R₄═CH₃ the benzoquinoid ansamycin is macbecin.

In another embodiment of the present invention the molecular chaperone inhibitor is radicicol.

In yet another embodiment the molecular chaperone inhibitor is novobiocin.

In yet another embodiment of the present invention the ansamycins are trienomycin and their analogues and derivatives. For example see U.S. Pat. No. 5,109,133.

The molecular chaperone inhibitors of the present invention are delivered, alone or in combination with synergistic and/or additive therapeutic agents, directly to the affected area using medical devices. Potentially synergistic and/or additive therapeutic agents may include drugs that impact a different aspect of the restenosis process such as antiplatelet, antimigratory or antifibrotic agents. Alternately they may include drugs that also act as antiproliferatives and/or antiinflammatories but through a different mechanism than inhibiting molecular chaperone activity. For example, and not intended as a limitation, synergistic combination considered to within the scope of the present invention include at least one molecular chaperone inhibitor and an antisense anti-c-myc oligonucleotide, at least one molecular chaperone inhibitor and rapamycin or analogues and derivatives thereof such a 40-0-(2-hydroxyethyl)-rapamycin, at least one molecular chaperone inhibitor and exochelin, at least one molecular chaperone inhibitor and n-acetyl cysteine inhibitors, at least one molecular chaperone inhibitor and a PPARγ agonist, and so on.

The medical devices used in accordance with the teachings of the present invention may be permanent medical implants, temporary implants, or removable devices. For examples, and not intended as a limitation, the medical devices of the present invention may include, stents, catheters, micro-particles, probes and vascular grafts.

In one embodiment of the present invention stents are used as the drug delivery platform. The stents may be vascular stents, urethral stents, biliary stents, or stents intended for use in other ducts and organ lumens. Vascular stents may be used in peripheral, neurological or coronary applications. The stents may be rigid expandable stents or pliable self expanding stents. Any biocompatible material may be used to fabricate the stents of the present invention including, without limitation, metals or polymers. The stents of the present invention may also be bioresorbable.

In one embodiment of the present invention vascular stents are implanted into coronary arteries immediately following angioplasty. However, one significant problem associated with stent implantation, specifically vascular stent deployment, is restenosis. Restenosis is a process whereby a previously opened lumen is re-occluded by VSMC proliferation. Therefore, it is an object of the present invention to provide stents that suppress or eliminate VSMC migration and proliferation and thereby reduce, and/or prevent restenosis.

In one embodiment of the present invention metallic vascular stents are coated with one or more anti-restenotic compound, specifically at least one molecular chaperone inhibitor, more specifically the molecular chaperone inhibitor is a benzoquinone ansamycin. The benzoquinone ansamycin may be dissolved or suspended in any carrier compound that provides a stable composition that does not react adversely with the device to be coated or inactivate the benzoquinone ansamycin. The metallic stent is provided with a biologically active benzoquinone ansamycin coating using any technique known to those skilled in the art of medical device manufacturing. Suitable non-limiting examples include impregnation, spraying, brushing, dipping and rolling. After the benzoquinone ansamycin solution is applied to the stent it is dried leaving behind a stable benzoquinone ansamycin delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the benzoquinone ansamycin compositions of the present invention.

The anti-restenotic effective amounts of molecular chaperone inhibitors used in accordance with the teachings of the present invention can be determined by a titration process. Titration is accomplished by preparing a series of stent sets. Each stent set will be coated, or contain different dosages of the molecular chaperone inhibitor agonist selected. The highest concentration used will be partially based on the known toxicology of the compound. The maximum amount of drug delivered by the stents made in accordance with the teaching of the present invention will fall below known toxic levels. Each stent set will be tested in vivo using the preferred animal model as described in Example 5 below. The dosage selected for further studies will be the minimum dose required to achieve the desired clinical outcome. In the case of the present invention, the desired clinical outcome is defined as the inhibition of vascular re-occlusion, or restenosis. Generally, and not intended as a limitation, an anti-restenotic effective amount of the molecular chaperone inhibitors of the present invention will range between about 0.5 ng to 1.0 mg depending on the particular molecular chaperone inhibitor used and the delivery platform selected.

In addition to the molecular chaperone inhibitor selected, treatment efficacy may also be affected by factors including dosage, route of delivery and the extent of the disease process (treatment area). An effective amount of a molecular chaperone inhibitor composition can be ascertained using methods known to those having ordinary skill in the art of medicinal chemistry and pharmacology. First the toxicological profile for a given molecular chaperone inhibitor composition is established using standard laboratory methods. For example, the candidate molecular chaperone inhibitor composition is tested at various concentration in vitro using cell culture systems in order to determine cytotoxicity. Once a non-toxic, or minimally toxic, concentration range is established, the molecular chaperone inhibitor composition is tested throughout that range in vivo using a suitable animal model. After establishing the in vitro and in vivo toxicological profile for the molecular chaperone inhibitor compound, it is tested in vitro to ascertain if the compound retains antiproliferative activity at the non-toxic, or minimally toxic ranges established.

Finally, the candidate molecular chaperone inhibitor composition is administered to treatment areas in humans in accordance with either approved Food and Drug Administration (FDA) clinical trial protocols, or protocol approved by Institutional Review Boards (IRB) having authority to recommend and approve human clinical trials for minimally invasive procedures. Treatment areas are selected using angiographic techniques or other suitable methods known to those having ordinary skill in the art of intervention cardiology. The candidate molecular chaperone inhibitor composition is then applied to the selected treatment areas using a range of doses. Preferably, the optimum dosages will be the highest non-toxic, or minimally toxic concentration established for the molecular chaperone inhibitor composition being tested. Clinical follow-up will be conducted as required to monitor treatment efficacy and in vivo toxicity. Such intervals will be determined based on the clinical experience of the skilled practitioner and/or those established in the clinical trial protocols in collaboration with the investigator and the FDA or IRB supervising the study.

The molecular chaperone inhibitor therapy of the present invention can be administered directly to the treatment area using any number of techniques and/or medical devices. In one embodiment of the present invention the molecular chaperone inhibitor composition is applied to a vascular stent. The vascular stent can be of any composition or design. For example, the sent may be self-expanding or mechanically expanded stent 10 using a balloon catheter FIG. 2. The stent 10 may be made from stainless steel, titanium alloys, nickel alloys or biocompatible polymers. Furthermore, the stent 10 may be polymeric or a metallic stent coated with at least one polymer. In other embodiments the delivery device is an aneurysm shield, a vascular graft or surgical patch. In yet other embodiments the molecular chaperone inhibitor therapy of the present invention is delivered using a porous or “weeping” catheter to deliver a molecular chaperone inhibitor containing hydrogel composition to the treatment area. Still other embodiments include microparticles delivered using a catheter or other intravascular or transmyocardial device.

In another embodiment an injection catheter can be used to deliver the chaperone inhibitors of the present invention either directly into, or adjacent to, a vascular occlusion or a vasculature site at risk for developing restenosis (treatment area). As used herein, adjacent means a point in the vasculature either distal to, or proximal from a treatment area that is sufficiently close enough for the anti-restenotoic composition to reach the treatment area at therapeutic levels. A vascular site at risk for developing restenosis is defined as a treatment area where a procedure is conducted that may potentially damage the luminal lining. Non-limiting examples of procedures that increase the risk of developing restenosis include angioplasty, stent deployment, vascular grafts, ablation therapy, and brachytherapy.

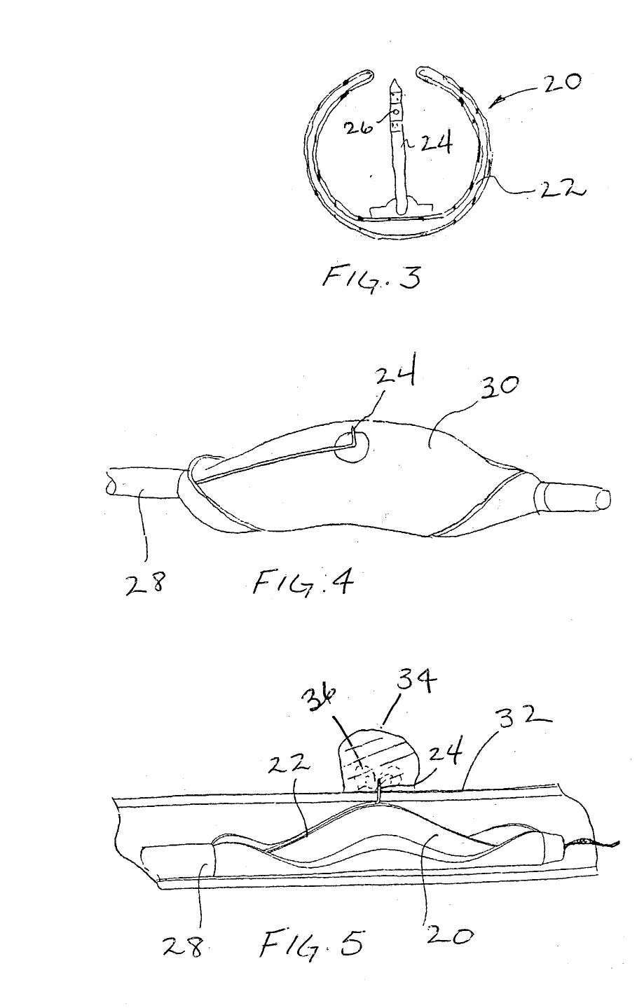

In one embodiment of the present invention an injection catheter as depicted in U.S. patent application publication Ser. No. 2002/0198512 A1 and related U.S. patent application Ser. Nos. 09/961,080, and 09/961,079 can be used to administer the chaperone inhibitors of the present invention directly to the adventia. FIGS. 3, 4 and 5 depict one such embodiment. FIG. 3 illustrates the C-shaped configuration of the catheter balloon 20 prior to inflation having the injection needle 24 nested therein and a balloon interior 22 connected to an inflation source (not shown) which permits the catheter body to be expanded as shown in FIG. 4. Needle 24 has an injection port 26 that transits the chaperone inhibitor into the adventia from a proximal reservoir (not shown) located outside the patient.

FIG. 4 illustrates the inflated balloon 30 attached to the catheter body 28 and injection needle 24 capable of penetrating the adventia. FIG. 5 depicts deployment of the chaperone inhibitor of the present invention directly into the adventia 34. The injection needle 24 penetrates the blood vessel wall 32 as balloon 20 is inflated and injects the chaperone inhibitor 36 into the tissue.

The medical device can be made of virtually any biocompatible material having physical properties suitable for the design. For example, tantalum, stainless steel and nitinol have been proven suitable for many medical devices and could be used in the present invention. Also, medical devices made with biostable or bioabsorbable polymers can be used in accordance with the teachings of the present invention. Although the medical device surface should be clean and free from contaminants that may be introduced during manufacturing, the medical device surface requires no particular surface treatment in order to retain the coating applied in the present invention. Both surfaces (inner 14 and outer 12 of stent 10, or top and bottom depending on the medical devices' configuration) of the medical device may be provided with the coating according to the present invention.

In order to provide the coated medical device according to the present invention, a solution which includes a solvent, a polymer dissolved in the solvent and a molecular chaperone inhibitor composition dispersed in the solvent is first prepared. It is important to choose a solvent, a polymer and a therapeutic substance that are mutually compatible. It is essential that the solvent is capable of placing the polymer into solution at the concentration desired in the solution. It is also essential that the solvent and polymer chosen do not chemically alter the molecular chaperone inhibitor's therapeutic character. However, the molecular chaperone inhibitor composition only needs to be dispersed throughout the solvent so that it may be either in a true solution with the solvent or dispersed in fine particles in the solvent. The solution is applied to the medical device and the solvent is allowed to evaporate leaving a coating on the medical device comprising the polymer(s) and the molecular chaperone inhibitor composition.

Typically, the solution can be applied to the medical device by either spraying the solution onto the medical device or immersing the medical device in the solution. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the medical device. In either a coating applied by spraying or by immersion, multiple application steps are generally desirable to provide improved coating uniformity and improved control over the amount of molecular chaperone inhibitor composition to be applied to the medical device. The total thickness of the polymeric coating will range from approximately 1 micron to about 20 microns or greater. In one embodiment of the present invention the molecular chaperone inhibitor composition is contained within a base coat, and a top coat is applied over the molecular chaperone inhibitor containing base coat to control release of the molecular chaperone inhibitor into the tissue.

The polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polyddioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones, polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer-to-molecular chaperone inhibitor composition ratio will depend on the efficacy of the polymer in securing the molecular chaperone inhibitor composition onto the medical device and the rate at which the coating is to release the molecular chaperone inhibitor composition to the tissue of the blood vessel. More polymer may be needed if it has relatively poor efficacy in retaining the molecular chaperone inhibitor composition on the medical device and more polymer may be needed in order to provide an elution matrix that limits the elution of a very soluble molecular chaperone inhibitor composition. A wide ratio of therapeutic substance-to-polymer could therefore be appropriate and could range from about 0.1% to 99% by weight of therapeutic substance-to-polymer.

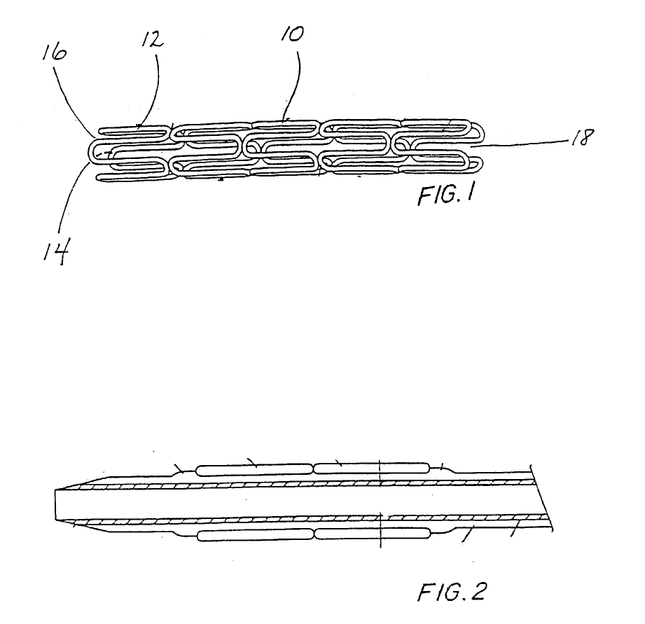

In one embodiment of the present invention a vascular stent as depicted in FIG. 1 is coated with molecular chaperone inhibitors using a two-layer biologically stable polymeric matrix comprised of a base layer and an outer layer. Stent 10 has a generally cylindrical shape and an outer surface 12, an inner surface 14, a first open end 16, a second open end 18 and wherein the outer and inner surfaces 12, 14 are adapted to deliver an anti-restenotic effective amount of at least one molecular chaperone inhibitor in accordance with the teachings of the present invention. Briefly, a polymer base layer comprising a solution of ethylene-co-vinylacetate and polybutylmethacrylate is applied to stent 10 such that the outer surface 12 is coated with polymer. In another embodiment both the inner surface 14 and outer surface 12 of stent 10 are provided with polymer base layers. The molecular chaperone inhibitor or mixture thereof is incorporated into the base layer. Next, an outer layer comprising only polybutylmethacrylate is applied to stent's 10 outer layer 14 that has been previous provide with a base layer. In another embodiment both the inner surface 14 and outer surface 12 of stent 10 are proved with polymer outer layers.

The thickness of the polybutylmethacrylate outer layer determines the rate at which the molecular chaperone inhibitors elute from the base coat by acting as a diffusion barrier. The ethylene-co-vinylacetate, polybutylmethacrylate and molecular chaperone inhibitor solution may be incorporated into or onto a medical device in a number of ways. In one embodiment of the present invention the molecular chaperone inhibitor/polymer solution is sprayed onto the stent 10 and then allowed to dry. In another embodiment, the solution may be electrically charged to one polarity and the stent 10 electrically changed to the opposite polarity. In this manner, the molecular chaperone inhibitor/polymer solution and stent will be attracted to one another thus reducing waste and providing more control over the coating thickness.

In another embodiment of the present invention the molecular chaperone inhibitor is a benzoquinone ansamycin and the polymer is bioresorbable. The bioresorbable polymer-benzoquinone ansamycin blends of the present invention can be designed such that the polymer absorption rate controls drug release. In one embodiment of the present invention a polycaprolactone-geldanamycin blend is prepared. A stent 10 is then stably coated with the polycaprolactone-geldanamycin blend wherein the stent coating has a thickness of between approximately 0.1 μm to approximately 100 μm. The polymer coating thickness determines the total amount of geldanamycin delivered and the polymer's absorption rate determines the administrate rate.

Using the preceding examples it is possible for one of ordinary skill in the part of polymer chemistry to design coatings having a wide range of dosages and administration rates. Furthermore, drug delivery rates and concentrations can also be controlled using non-polymer containing coatings and techniques known to persons skilled in the art of medicinal chemistry and medical device manufacturing,

The following examples are provided to more precisely define and enable the molecular chaperone inhibitor-eluting medical devices of the present invention. It is understood that there are numerous other embodiments and methods of using the present invention that will be apparent embodiments to those of ordinary skill in the art after having read and understood this specification and examples. Moreover, it is understood that benzoquinone ansamycins, specifically geldanamycin, is but one example of the molecular chaperone inhibitors that can be used according to the teachings of the present invention. These alternate embodiments are considered part of the present invention.

### EXAMPLE 1

Metal Stent Cleaning Procedure

Stainless steel stents were placed a glass beaker and covered with reagent grade or better hexane. The beaker containing the hexane immersed stents was then placed into an ultrasonic water bath and treated for 15 minutes at a frequency of between approximately 25 to 50 KHz. Next the stents were removed from the hexane and the hexane was discarded. The stents were then immersed in reagent grade or better 2-propanol and vessel containing the stents and the 2-propanol was treated in an ultrasonic water bath as before. Following cleaning the stents with organic solvents, they were thoroughly washed with distilled water and thereafter immersed in 1.0 N sodium hydroxide solution and treated at in an ultrasonic water bath as before. Finally, the stents were removed from the sodium hydroxide, thoroughly rinsed in distilled water and then dried in a vacuum oven over night at 40° C.

After cooling the dried stents to room temperature in a desiccated environment they were weighed their weights were recorded.

### EXAMPLE 2

Coating a Clean, Dried Stent Using a Drug/Polymer System

250 μg of geldanamycin was carefully weighed and added to a small neck glass bottle containing 27.56 ml of tetrahydofuran (THF). The geldanamycin-THF suspension was then thoroughly mixed until a clear solution is achieved.

Next 251.6 mg of polycaprolactone (PCL) was added to the geldanamycin-THF solution and mixed until the PCL dissolved forming a drug/polymer solution.

The cleaned, dried stents were coated using either spraying techniques or dipped into the drug/polymer solution. The stents were coated as necessary to achieve a final coating weight of between approximately 10 μg to 1 mg. Finally, the coated stents were dried in a vacuum oven at 50° C. over night. The dried, coated stents were weighed and the weights recorded.

The concentration of drug loaded onto (into) the stents was determined based on the final coatingweight. Final coating weight is calculated by subtracting the stent's pre-coating weight from the weight of the dried, coated stent.

### EXAMPLE3

Coating a Clean, Dried Stent Using a Sandwich-Type Coating

In one embodiment of the present invention a cleaned, dry stent was first coated with polyvinyl pyrrolidone (PVP) or another suitable polymer followed by a coating of geldanamycin. Finally, a second coating of PVP was provided to seal the stent thus creating a PVP-geldanamycin-PVP sandwich coated stent. In another embodiment a parylene primer is applied to the bare metal stent prior to applying the geldanamycin-containing polymer coating. In yet another embodiment, a polymer cap coat is applied over the geldanamycin coating wherein the cap coat comprises a different polymer from the polymer used in the geldanamycin-containing polymer coating.

In another embodiment of the present invention a polybutylmethacrylate-polyethylene vinyl acetate polymer blend was used to control the release of geldanamycin.

The following example is not intended as a limitation but only as one possible polymer coating that can be used in accordance with the teachings of the present invention. Other coatings will be discussed herein and are considered within the scope of the present invention.

The Sandwich Coating Procedure: 100 mg of PVP was added to a 50 mL Erlenmeyer containing 12.5 ml of THF. The flask was carefully mixed until all of the PVP is dissolved. In a separate clean, dry Erlenmeyer flask 250 μg of geldanamycin was added to 11 mL of THF and mixed until dissolved.

A clean, dried stent was then sprayed with PVP until a smooth confluent polymer layer was achieved. The stent was then dried in a vacuum oven at 50° C. for 30 minutes.

Next the nine successive layers of the geldanamycin were applied to the polymer-coated stent. The stent was allowed to dry between each of the successive geldanamycin coats. After the final geldanamycin coating had dried, three successive coats of PVP were applied to the stent followed by drying the coated stent in a vacuum oven at 50° C. over night. The dried, coated stent is weighed and its weight recorded.

The concentration of drug in the drug/polymer solution and the final amount of drug loaded onto the stent determine the final coating weight. Final coating weight is calculated by subtracting the stent's pre-coating weight from the weight of the dried, coated stent.

### EXAMPLE 4

Coating a Clean, Dried Stent with Pure Drug

1.00 μg of geldanamycin was carefully weighed and added to a small neck glass bottle containing 11.4 ml of absolute methanol (MeOH). The geldanamycin-Methanol suspension was then heated at 50° C. for 15 minutes and then mixed until the geldanamycin was completely dissolved.

Next a clean, dried stent was mounted over the balloon portion of angioplasty balloon catheter assembly. The stent was then sprayed with, or in an alternative embodiment, dipped into, the geldanamycin-MeOH solution. The coated stent was dried in a vacuum oven at 50° C. over night. The dried, coated stent was weighed and its weight recorded.

The concentration of drug loaded onto (into) the stents was determined based on the final coating weight. Final coating weight is calculated by subtracting the stent's pre-coating weight from the weight of the dried, coated stent.

### EXAMPLE 5

In vivo Testing of a Molecular Chaperone Inhibitor-Coated Vascular Stent in a Porcine Model

The ability of a molecular chaperone inhibitor γ agonist to reduce neointimal hyperplasia in response to intravascular stent placement in an acutely injured porcine coronary artery is demonstrated in the following example. Two controls and three treatment arms were used as outlined below:

1. Control Groups:

Six animals were used in each control group. The first control group tests the anti-restenotic effects of the clean, dried MedtronicAVE S7 stents having neither polymer nor drug coatings. The second control group tests the anti-restenotic effects of polymer alone. Clean, dried MedtronicAVE S7 stents having polybutylmethacrylate-polyethylene vinyl acetate polymer blend coatings without drug were used in the second control group.

2. Experimental Treatment Groups

Three different stent configurations and two different drug dosages are evaluated for their anti-restenotic effects. Twelve animals are included in each group.

Group 1 MedtronicAVE S7 stents having a coating comprised of a 75:25 polybutylmethacrylate-polyethylene vinyl acetate polymer blend containing 10% geldanamycin by weight are designated the fast release group in accordance with the teachings of the present invention.

Group 2 MedtronicAVE S7 stents having a coating comprised of a 80:20 polybutylmethacrylate-polyethylene vinyl acetate polymer blend containing 10% geldanamycin by weight are designated the slow release group in accordance with the teachings of the present invention.

The swine has emerged as the most appropriate animal model for the study of the endovascular devices. The anatomy and size of the coronary vessels are comparable to that of humans. Furthermore, the neointimal hyperplasia that occurs in response to vascular injury is similar to that seen clinically in humans. Results obtained in the swine animal model are considered predictive of clinical outcomes in humans. Consequently, regulatory agencies have deemed six-month data in the porcine sufficient to allow progression to human trials. Therefore, as used herein “animal” shall include mammals, fish, reptiles and birds. Mammals include, but are not limited to, primates, including humans, dogs, cats, goats, sheep, rabbits, pigs, horses and cows.

Non-atherosclerotic acutely injured RCA, LAD, and/or LCX arteries of the Farm Swine (or miniswine) are utilized in this study. Placement of coated and control stents is random by animal and by artery. The animals are handled and maintained in accordance with the requirements of the Laboratory Animal Welfare Act (P.L.89-544) and its 1970 (P.L. 91-579), 1976 (P.L. 94-279), and 1985 (P.L. 99-198) amendments. Compliance is accomplished by conforming to the standards in the Guide for the Care and the Use of Laboratory Animals, ILAR, National Academy Press, revised 1996. A veterinarian performs a physical examination on each animal during the pre-test period to ensure that only healthy pigs are used in this study.

A. Pre-Operative Procedures

The animals were monitored and observed 3 to 5 days prior to experimental use. The animals had their weight estimated at least 3 days prior to the procedure in order to provide appropriate drug dose adjustments for body weight. At least one day before stent placement, 650 mg of aspirin is administered. Animals are fasted twelve hours prior to the procedure.

B. Anesthesia

Anesthesia was induced in the animal using intramuscular Telazol and Xylazine. Atropine is administered (20 μg/kg I.M.) to control respiratory and salivary secretions. Upon induction of light anesthesia, the subject animal was intubated. Isoflurane (0.1 to 5.0% to effect by inhalation) in oxygen is administered to maintain a surgical plane of anesthesia. Continuous electrocardiographic monitoring was performed. An I.V. catheter was placed in the ear vein in case it is necessary to replace lost blood volume. The level of anesthesia is monitored continuously by ECG and the animal's response to stimuli.

C. Catheterization and Stent Placement

Following induction of anesthesia, the surgical access site was shaved and scrubbed with chlorohexidine soap. An incision was made in the region of the right or left femoral (or carotid) artery and betadine solution was applied to the surgical site. An arterial sheath was introduced via an arterial stick or cutdown and the sheath was advanced into the artery. A guiding-catheter was placed into the sheath and advanced via a 0.035″ guide wire as needed under fluoroscopic guidance into the ostium of the coronary arteries. An arterial blood sample was obtained for baseline blood gas, ACT and HCT. Heparin (200 units/kg) is administered as needed to achieve and maintain ACT≧300 seconds. Arterial blood pressure, heart rate, and ECG are recorded.

After placement of the guide catheter into the ostium of the appropriate coronary artery, angiographic images of the vessels are obtained in at least two orthagonal views to identify the proper location for the deployment site. Quantitative coronary angiography (QCA) is performed and recorded. Nitroglycerin (200 μg I.C.) may be administered prior to treatment and as needed to control arterial vasospasm. The delivery system was prepped by aspirating the balloon with negative pressure for five seconds and by flushing the guidewire lumen with heparinized saline solution.

Deployment, patency and positioning of stent were assessed by angiography and a TIMI score is recorded. Results are recorded on video and cine. Final lumen dimensions are measured with QCA and/or IVUS. These procedures are repeated until a device was implanted in each of the three major coronary arteries of the pig. The stents were deployed haing an expansion ratio of 1:1.2. After final implant, the animal is allowed to recover from anesthesia. Aspirin is administered at 325 mg p.o. qd until sacrificed 28 days later.

D. Follow-Up Procedures and Termination

After 28 days, the animals were anesthetized and a 6F arterial sheath was introduced and advanced. A 6F large lumen guiding-catheter (diagnostic guide) was placed into the sheath and advanced over a guide wire under fluoroscopic guidance into the coronary arteries. After placement of the guide catheter into the appropriate coronary ostium, angiographic images of the vessel are taken to evaluate the stented. sites. At the end of the re-look procedure, the animals were euthanized with an overdose of Pentabarbitol I.V. and KCL I.V. The heart, kidneys, and liver are harvested and visually examined for any external or internal trauma. The organs were flushed with 1000 ml of lactated ringers at 100 mmHg and then flushed with 1000 ml of formalin at 100-120 mmHg. All organs are stored in labeled containers of formalin solution.

E. Histology and Pathology

The stented vessels were X-rayed prior to histology processing. The stented segments were processed for routine histology, sectioned, and stained following standard histology lab protocols. Appropriate stains were applied in alternate fashion on serial sections through the length of the treated vessels.

F. Data Analysis and Statistics

1. QCA Measurement

Quantitative angiography was performed to measure the balloon size at peak inflation as well as vessel diameter pre- and post-stent placement and at the 28 day follow-up. The following data are measured or calculated from angiographic data:

Stent-to-artery-ratio

Minimum lumen diameter (MLD)

Distal and proximal reference lumen diameter

Percent Stenosis=(Minimum lumen diameter÷reference lumen diameter)×100

2. Histomorphometric Analysis

Histologic measurements were made from sections from the native proximal and distal vessel and proximal, middle, and distal portions of the stent. A vessel injury score was calculated using the method described by Schwartz et al. (Schwartz R S et al. Restenosis and the proportional neointimal response to coronary artery injury: results in a porcine model. J Am Coll Cardiol 1992; 19:267-74). The mean injury score for each arterial segment was calculated. Investigators scoring arterial segment and performing histopathology were “blinded” to the device type. The following measurements are determined:

External elastic lamina (EEL) area

Internal elastic lamina (IEL) area

Luminal area

Adventitial area

Mean neointimal thickness

Mean injury score

3. The Neointimal Area and the % of In-Stent Restenosis are Calculated as Follows:

Neointimal area=(IEL−luminal area)

In-stent restenosis=[1−(luminal area÷IEL)]×100.

A given treatment arm is deemed beneficial if treatment results in a significant reduction in neointimal area and/or in-stent restenosis compared to both the bare stent control and the polymer-on control.

G. Surgical Supplies and Equipment

The following surgical supplies and equipment are required for the procedures described above:

1. Standard vascular access surgical tray

2. Non-ionic contrast solution

3. ACT machine and accessories

4. HCT machine and accessories (if applicable)

5. Respiratory and hemodynamic monitoring system

6. IPPB Ventilator, associated breathing circuits and Gas Anesthesia Machine

7. Blood gas analysis equipment

8. 0.035″ HTF or Wholey modified J guidewire, 0.014″ Guidewires

9. 6, 7, 8, and 9F introducer sheaths and guiding catheters (as applicable)

10. Cineangiography equipment with QCA capabilities

11. Ambulatory defibrillator

12. Standard angioplasty equipment and accessories

13. IVUS equipment (if applicable)

14. For radioactive labeled cell studies (if applicable):

15. Centrifuge

16. Aggregometer

17. Indium 111 oxime or other as specified

18. Automated Platelet Counter

19. Radiation Detection Device

F. Results

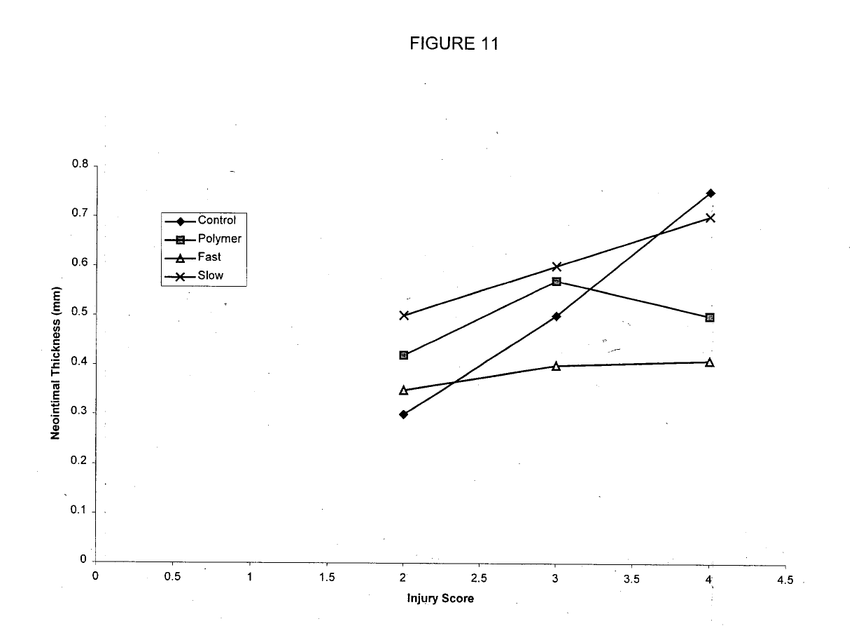

The results of the animal experiments are depicted in FIG. 11. FIG. 11 graphically depicts 28-day efficacy studies in farm swine. Medtroinc S7 stents (18 mm×3-3.51 mm diameter) were coated as described herein were sterilized and implanted into farm swine at an expansion ratio of 1:1.2 as described above. Animals were allowed to recover, and held for 28 d, after which the animal was euthanized and the tissue fixed and processed for histochemistry and histomorphometry, using standard techniques. FIG. 11. graphically depicts the correlation between neointimal thickness and injury score in the combined proximal and distal stent segments. The neointimal thickness and injury score were measured at each strut of the stent. A good correlation was observed between the injury score and neointimal thickness in the bare stent control group. A significant decrease in the neointimal thickness when the injury score increases was observed when the data from the “fast-release” stent is compared with the “slow-release” and bare stent controls. In FIG. 11 solid diamonds depict the bare metal MedtronicAVE S7 control stent; squares depict MedtronicAVE S7 contol stents having )a polymer-only coating (no drug); triangles depict MedtronicAVE S7 stents having the “fast elution profile” coatings and diamonds depict MedtronicAVE S7 stents having the “slow elution profile” coatings. These results clearly demonstrate the fast release geldanamycin containing coatings provide stents having reduced mean injury scores when compared to the controls.

### EXAMPLE 6

Inhibition of Human Coronary Artery Smooth Muscle Cells by Geldanamycin

A. Materials

1. Human coronary smooth muscles cells (HCASMC) were obtained from Clonetics, a division of Cambrex, Inc.

2. HCASMA basal media, supplied by Clonetics and supplemented with fetal bovine serum, insulin, hFGF-B (human fibroblast growth factor) hEGF (human epidermal growth factor).

3. Geldanamycin Sigma Chemical Company (Europe)

4. Absolute methanol

5. Twenty-four well polystyrene tissue culture plates

B. Human Coronary Artery Smooth Muscle Cells Proliferation Inhibition Studies.

Human coronary smooth muscles cells (HCASMC) were seeded in 24 well polystyrene tissue culture plates at a density of 5×10³ cells per well. Two different feeding and reading strategies were employed. Strategy 1: Cells were plated in cell culture media containing various concentrations of geldanamycin (see Table 1) and incubated at 37° C. for 48 hours. After the initial 48 hour incubation, the geldanamycin containing plating media was changed and the cells were fed with drug free media and incubated for an additional 48 hours and then read.

Strategy 2: Cells were plated in cell culture media containing various concentrations of geldanamycin (see Table 1) and incubated at 37° C. for 48 hours. After the initial 48 hours incubation, the geldanamycin-containing plating media was changed and the cells were fed with geldanamycin-containing media and incubated for an additional 48 hours and then read.

A 0.5 mg/mL stock solution of Geldanamycin was prepared in absolute methanol and diluted to the following final test concentrations in cell culture media:

TABLE 1
Test Concentrations of Geldanamycin used in vitro.

| nM Geldanamycin | ng/ml Geldanamycin |
|---|---|
| 0 | 0 |
| 0.1 | 0.06 |
| 0.5 | 0.28 |
| 1 | 0.56 |
| 5 | 2.8 |
| 10 | 5.61 |
| 50 | 28.03 |
| 100 | 56.06 |

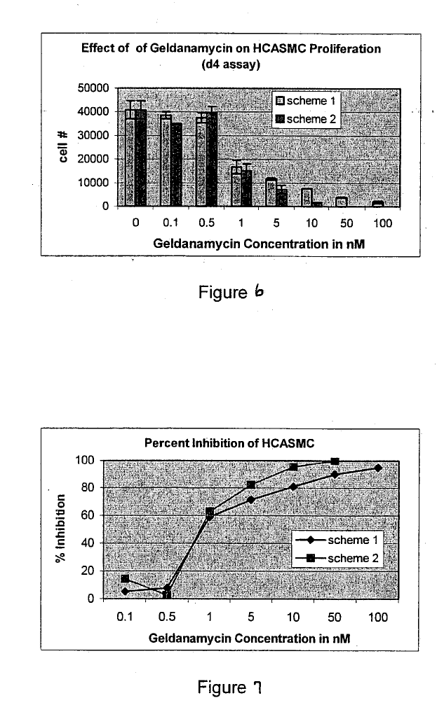

On day four cultures were analyzed to determine the proliferation inhibition effects of geldanamycin. FIGS. 6 and 7 graphically depict the percent inhibition at geldanamycin levels between 0.1 nM to 100 nM for both cell culture schemes. It can be seen from FIGS. 6 and 7 that significant HCASMC inhibition (>50% inhibition) begins at a dosage of 0.9 nM and rises dramatically to nearly 100% at 50 nM.

### EXAMPLE 7

Drug Elusion Profiles of Geldanamycin from Coated Stents

Vascular stents such as, but not limited to MedtronicAVE S670, S660 and S7 were provided with polymer coatings containing geldanamycin and the elusion profiles determined.

In vitro Drug Elution Studies

A. Fast Geldanamycin Eluting Coating

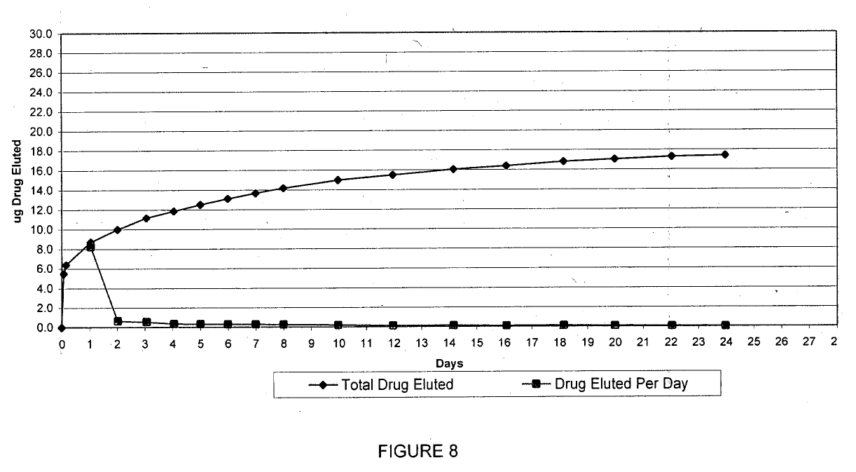

An 18.0 mm long×3.0 mm diameter stent was provided with a drug eluting polymer coating as described above. In this example the coating comprised a 75:25 polybutylmethacrylate-polyethylene vinyl acetate polymer blend containing 10% geldanamycin by weight. The coated stents were incubated in 2 mL of elution media (0.4% SDS in 10 mM Tris, pH6) that was pre-warmed to 37 C. The elution media was collected daily and replaced with 2 ml of pre-warmed elution media. The drug content was analyzed by HPLC using a water: acetonitrile gradient on a Waters NovaPack C18 column with detectection by UV at 304 nm wavelength. The elution profile depicted in FIG. 8 is a “fast elution” rate.

B. Slow Geldanamycin Eluting Coating

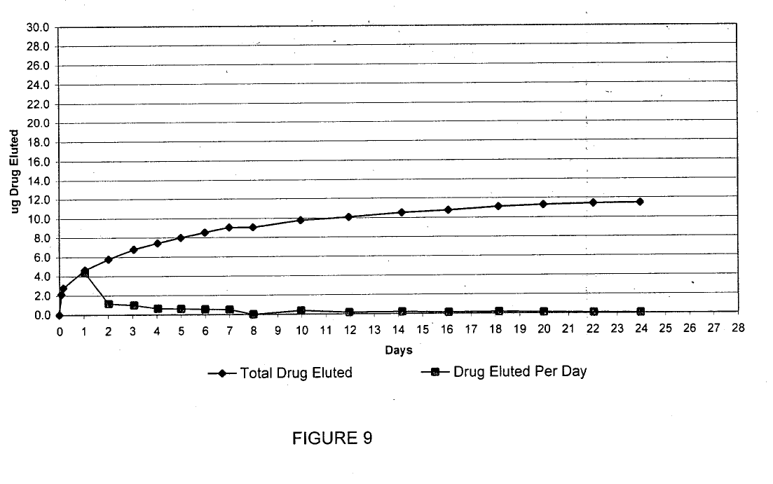

In another in vitro drug elution experiment an 18.0 mm long×3.0 mm diameter stent was provided with a drug eluting polymer coating comprised of an 80:20 polybutylmethacrylate-polyethylene vinyl acetate polymer blend containing 10% geldanamycin by weight. The coated stents were incubated in 2 mL of elution media (0.4% SDS in 10 mM Tris, pH6) that was pre-warmed to 37 C. The elution media was collected daily and replaced with 2 ml of pre-warmed elution media. The drug content was analyzed by HPLC using a water: acetonitrile gradient on a Waters NovaPack C18 column with detection by UV at 304 nm wavelength. The elution profile depicted in FIG. 9 is a “slow elution” rate.

In vivo Drug Elution Studies

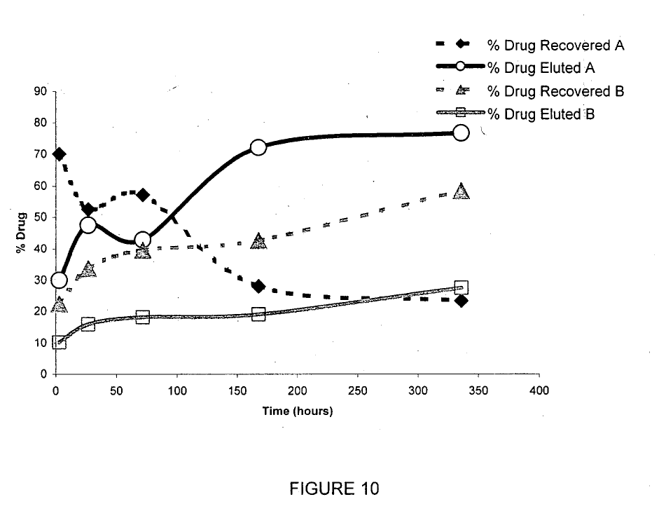

For in vivo studies stents having both fast and slow geldanamycin eluting coatings were prepare as described above. The coated stents were implanted into rabbit iliacs for a total of 336 hrs. At each time point depicted in FIG. 10 rabbits were euthanized and the stented vessels removed and reserved. After all stents were recovered from all time points the tissue around each stent was carefully removed, and the stents were incubated at 37 C. in dimethylsulfoxide (DMSO) until the remaining coating was stripped from the stent surface. The drug content of the DMSO was analyzed using HPLC as described above. The concentration of the drug remaining in the coating after removal from the rabbit iliac is inversely proportional to the total amount of drug eluted in vivo for a given time point. For comparison purposes stents prepared identically to those used in vivo were incubated in elution buffer as described above and tested in parallel with the in vivo stents at each time point.

FIG. 10 graphically compares in vivo drug elution profiles with their corresponding in vitro drug elution profiles. In vivo drug elution profiles are depicted in dashed lines; in vitro drug elution profiles are depicted in solid lines. Stents having the “slow elution rate” coatings are represent by triangles for in vivo studies and open boxes for in vitro tests. “Fast elution rate” coatings are represent by diamonds for in vivo studies and open circles for in vitro tests.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A medical device for delivering an anti-restenotic composition comprising:
a stent having a generally cylindrical shape comprising an outer surface, an inner surface, a first open end, a second open end and wherein at least one of said inner or said outer surfaces are adapted to deliver an anti-restenotic effective amount of at least one molecular chaperone inhibitor to a tissue within a mammal.

2. The medical device according to claim 1 wherein said stent is mechanically expandable.

3. The medical device according to claim 1 wherein said stent is self expandable.

4. The medical device according to claim 1 wherein said at least one molecular chaperone inhibitor is present on both said inner surface and said outer surface of said stent.

5. The medical device according to claim 1 wherein at least one of said inner or said outer surfaces are coated with a polymer wherein said polymer has at least one molecular chaperone inhibitor incorporated therein and said polymer releases said at least one molecular chaperone inhibitor into said tissue of said mammal.

6. The medical device according to claim 1 wherein said at least one molecular chaperone inhibitor inhibits or interferes with the normal biological function of a heat shock protein.

7. The medical device according to claim 6 wherein said at least one molecular chaperone inhibitor is a benzoquinoid ansamycin.

8. The medical device according to claim 7 wherein said benzoquinoid ansamycin is selected from the group consisting of geldanamycin, herbimycin, macbecin and derivatives and analogues thereof.

9. The medical device according to claim 1 wherein said stent is delivered to said tissue of said anatomical lumen using a balloon catheter.

10. The medical device according to claim 1 wherein said tissue is a blood vessel lumen.

11. The medical device according to claim 5 wherein said polymer is selected from the group consisting of polyurethanes, silicones, polyolefins, polyisobutylene, ethylene-alphaolefin copolymers, acrylic polymers and copolymers, ethylene-co-vinylacetate, polybutylmethacrylate, vinyl halide polymers and copolymers, polyvinyl chloride; polyvinyl ethers, polyvinyl methyl ether, polyvinylidene halides, polyvinylidene fluoride, polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile,styrene copolymers, ABS resins, and ethylene-vinyl acetate copolymers, polyamides, such as Nylon 66 and polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate; cellophane, cellulose nitrate, cellulose propionate, cellulose ethers, carboxymethyl cellulose and combinations thereof.

11. A vascular stent comprising a polymeric coating containing an anti-restenotic effective amount of a molecular chaperone inhibitor.

12. The vascular stent of claim 11 further comprising a parylene primer coat.

13. The vascular stent of claim 11 wherein said polymeric coating comprises a polybutylmethacrylate-polyethylene vinyl acetate polymer blend.

14. The vascular stent of claim 1 or claim 11 wherein said molecular chaperone inhibitor is in a concentration of between 0.1% to 99% by weight of molecular chaperone inhibitor-to-polymer.

15. The vascular stent according to claim 11 wherein said at least one molecular chaperone inhibitor inhibits or interferes with the normal biological function of a heat shock protein.

16. The vascular stent according to claim 11 wherein said at least one molecular chaperone inhibitor is a benzoquinoid ansamycin.

17. The vascular stent according to claim 16 wherein said benzoquinoid ansamycin is selected from the group consisting of geldanamycin, herbimycin, macbecin and derivatives and analogues thereof.

18. The vascular stent according to claim 11 wherein said stent is delivered to a tissue of a mammal's anatomical lumen using a balloon catheter.

19. A method for inhibiting restenosis in a mammal comprising the site specific delivery of at least one molecular chaperone inhibitor.

20. The method according to claim 19 wherein said molecular chaperone inhibitor is delivered to a site at risk for restenosis using a vascular stent.

21. The method according to claim 19 wherein said molecular chaperone inhibitor is delivered to a site at risk for restenosis using an injection catheter.

22. The method according to claim 19 wherein said at least one molecular chaperone inhibitor inhibits or interferes with the normal biological function of a heat shock protein.

23. The method according to claim 19 wherein said at least one molecular chaperone inhibitor is a benzoquinoid ansamycin.

24. The method according to claim 20 wherein said benzoquinoid ansamycin is selected from the group consisting of geldanamycin, herbimycin, macbecin and derivatives and analogues thereof.

25. The method according to claim 22 wherein the heat shock protein is selected from the group consisting of 100 kDa, 90 kDa, 70 kDa, 60 kDa, 40 kDa, 25 kDa, and 20 kDa molecular weight HSPs.

26. A method for inhibiting restenosis comprising providing a vascular stent having a coating comprising an anti-restenotic effective amount of geldanamycin.
